SEQUENCE LISTING (8 sequences)

SEQ ID NO: 1 - protein, 4 aa, artificial
  synthesized bitter taste blocker
TMTL

SEQ ID NO: 2 - protein, 4 aa, artificial
  synthesized bitter taste blocker
ETCL

SEQ ID NO: 3 - protein, 6 aa, artificial
  synthesized bitter taste blocker
SSMSSL

SEQ ID NO: 4 - protein, 7 aa, artificial
  synthesized bitter taste blocker
ETSARHL

SEQ ID NO: 5 - protein, 10 aa, artificial
  synthesized bitter taste blocker
AGDDAPRAVF

SEQ ID NO: 6 - protein, 4 aa, artificial
  synthesized bitter taste blocker
AAMY

SEQ ID NO: 7 - protein, 4 aa, artificial
  synthesized bitter taste blocker
VSSY

SEQ ID NO: 8 - protein, 4 aa, artificial
  synthesized bitter taste blocker
AAYM

(19) United States
(12) Patent Application Publication
(10) Pub. No.: US 2021/0045419 A1
(43) Pub. Date: Feb. 18, 2021

(54) Food Protein-Derived Peptides as Bitter Taste Blockers

(71) Applicant: UNIVERSITY OF MANITOBA, Winnipeg (CA)

(72) Inventors: Prashen Chelikani, Winnipeg (CA); Rotimi Aluko, Winnipeg (CA)

(21) Appl. No.: 16/969,395

(22) PCT Filed: Feb. 15, 2019
(86) PCT No.: PCT/CA2019/050186

§ 371 Date: Aug. 12, 2020

Related U.S. Application Data
(60) Provisional application No. 62/632,506, filed on Feb. 20, 2018.

(51) Int. Cl. A23L 27/21; A23J 3/34; A61K 47/42; C07K 7/06; C12P 21/06

(52) U.S. Cl. CPC A23L 27/21; A23J 3/34; C12P 21/06; C07K 7/06; A61K 47/42

(57) ABSTRACT

Beef protein was hydrolyzed with each of six commercial enzymes (alcalase, chymotrypsin, trypsin, pepsin, flavourzyme, and thermoase). Electronic tongue measurements showed that the hydrolysates had significantly (p<0.05) lower bitter scores than quinine. Addition of the hydrolysates to quinine led to reduced bitterness intensity of quinine with trypsin and pepsin hydrolysates being the most effective. Addition of the hydrolysates to HEK293T cells that heterologously express one of the bitter taste receptors (T2R4) showed alcalase, thermoase, pepsin and trypsin hydrolysates as the most effective in reducing calcium mobilization. Eight peptides that were identified from the alcalase and chymotrypsin hydrolysates also suppressed bitter agonist-dependent calcium release from T2R4 and T2R14 with AGDDAPRAVF and ETSARHL being the most effective.

### PRIOR APPLICATION INFORMATION

The instant application claims the benefit of U.S. Provisional Patent Application Ser. No. 62/632,506, filed Feb. 20, 2018 and entitled FOOD PROTEIN-DERIVED PEPTIDES AS BITTER TASTE BLOCKERS, the entire contents of which are incorporated herein by reference.

### BACKGROUND OF THE INVENTION

Taste is defined as a chemosensation arising from the oral cavity. The taste chemosensation is responsible for basic food appraisal and is mediated mostly by G protein-coupled receptors (GPCRs)^{52, 53}. With more than 700 GPCRs identified in the human genome, they form the largest known family of cell surface receptors^{54,55}. Humans are capable of detecting five basic tastes: sweet, umami, bitter, sour and salt.

Human beings are naturally averse to bitter taste because of the non-pleasant oral sensation coupled with the fact that some causative compounds are usually toxic and may be life-threatening.^{1,2} Therefore, eliminating or reducing the bitter taste attribute of pharmaceutical and food products could enhance organoleptic properties and consumer acceptance. Bitter taste is sensed by 25 bitter taste receptors (T2Rs), which are responsible for human perception of thousands of bitter-tasting substances.^{3,4} So far, only a few antagonists or blockers against specific T2Rs have been identified to work at the receptor level to reduce bitterness intensity of a limited number of food products^{5,6,7}, and these compounds have been extensively reviewed recently.⁸ Therefore, discovery of additional bitter blockers are required in order to expand coverage of several other foods that require bitter taste masking, especially for improved commercial success. A diversity of bitter taste-blocking compounds will also enable determination of structural properties that enhance interactions with T2Rs to block activation by bitter substances. However, due to the limited understanding of the mechanism of bitter taste signal transduction, progress towards production and utilization of new blockers has been slow. This is complicated by the fact that in addition to the oral cavity, T2Rs are also expressed in the brain, large intestine, testis, nasal epithelium and human airway.^{4,9} These extraoral T2Rs are believed to participate in various physiological functions and are considered potential therapeutic targets for diseases such as those that affect the respiratory airway, especially asthma-related dysfunctions.¹⁰⁻¹²

The most interesting question in bitter signaling research is how 25 T2Rs are capable of detecting hundreds of structurally diverse compounds. Some T2Rs are activated by a wide range of compounds, whereas some are activated by a single bitter compound⁵⁶⁻⁵⁹. For example, T2R31, T2R43 and T2R46 have approximately 85% sequence homology, but they bind to different agonists⁶⁰, giving credence to the hypothesis that each T2R might have a unique ligand binding pocket. Interestingly, T2R38 the highly studied taste receptor also referred to as the PTC or PROP receptor is very selective for Phenylthiocarbamide (PTC) and 6-n-propyl-2-thiouracil (PROP). Sensitivity to the bitter substances PTC/PROP is an inherited trait. Naturally occurring alleles of TAS2R38 gene are responsible for individual differences to taste PTC and PROP. Based on this, humans are classified into super-tasters, tasters and non-tasters.

Bioactive peptides (BAPs) that are generated from enzymatic hydrolysis of food proteins are increasingly gaining attention because they have been reported to possess multifunctional health-promoting properties that include antihypertensive, anti-inflammatory and anticancer.¹³⁻¹⁵ However, there is limited information on bitterness-suppressing properties of food protein hydrolysates and their constituent peptide chains. Previous studies have reported that amino acids and peptides have the capacity to efficiently diminish bitter taste intensity. For example, L-aspartyl-L-phenylalanine and L-ornithyl-L-alanine were reported to reduce the bitterness of potassium chloride.¹⁶ Furthermore, the simple nucleotides cytosine monophosphate (CMP) and 2-deoxyadenosine triphosphate (dATP) were demonstrated to cause a 40% and 60% reduction in bitterness of a 10 mM quinine solution, respectively.¹⁷ Besides, a mixture of amino acids (L-asparagine, L-methionine, L-tyrosine, L-serine, L-aspartic acid, L-glutamine, L-alanine, L-leucine, and L-proline) was reported to suppress the bitter after-taste of high potency sweeteners.¹⁸ However, quantitative data and the mechanisms of bitter taste suppression by amino acids and peptides remain scarce.

Food protein derived peptides have varied tastes ranging from sweet to bitter. This depends on the size, composition, and position of amino acids in the peptide sequence. Peptides with a bitter taste present in soybean paste, soy sauce, aged cheese and fermented products were reported to activate few of the 25 T2Rs^{3,35}. The use of active lactase to hydrolyze lactose during storage is a common process to produce lactose-hydrolyzed milk. Recently it was shown that bitter peptides were produced in lactose-hydrolyzed milk affecting its taste profile, when commercial lactase was used in the process⁶¹. However, no functional analysis of these food protein peptides or of protein hydrolysates was carried out on the majority of T2Rs, including the highly expressed T2Rs in human tissues: T2R4, T2R7, T2R10, T214 and T2R20.

The ligands that reduce the activity of T2Rs, which include both antagonists and inverse agonists, are referred to as bitter blockers⁸. Only 15 bitter blockers have been reported⁸. Interestingly, none of these blockers can block all the 25 T2Rs and some of them act as agonists on other T2Rs. As will be appreciated by one of skill in the art, the bitter taste is reduced if the majority of the receptors are blocked, as activating only one or two receptors would do not produce the same efficacy or taste sensation.

Beef proteins have been shown to generate (through enzymatic hydrolysis) desirable flavor-promoting peptides.^{19,20} Thus, we envisaged that beef could also be an excellent raw material to generate peptides with bitter taste-blocking properties. Therefore, the aim of this work was to determine the bitter taste-blocking ability of enzymatic meat protein hydrolysates followed by elucidation of the structural and functional properties of the main peptides.

### SUMMARY OF THE INVENTION

According to a first aspect of the invention, there is provided a bitter taste blocking peptide, said peptide consisting of an amino acid sequence selected from the group consisting of: TMTL (SEQ ID No:1); ETCL (SEQ ID No:2); SSMSSL (SEQ ID No:3); ETSARHL (SEQ ID No:4); AGDDAPRAVF (SEQ ID No:5); AAMY (SEQ ID No:6); VSSY (SEQ ID No:7); and AAYM (SEQ ID No:8).

In some embodiments, the bitter taste blocking peptide consists of the amino acid sequence AGDDAPRAVF (SEQ ID No:5) or ETSARHL (SEQ ID No:4).

In some embodiments, the bitter taste blocking peptide is a T2R4 receptor antagonist.

According to another aspect of the invention, there is provided a T2R receptor antagonist peptide, said peptide consisting of an amino acid sequence selected from the group consisting of: TMTL (SEQ ID No:1); ETCL (SEQ ID No:2); SSMSSL (SEQ ID No:3); ETSARHL (SEQ ID No:4); AGDDAPRAVF (SEQ ID No:5); AAMY (SEQ ID No:6); VSSY (SEQ ID No:7); and AAYM (SEQ ID No:8).

In some embodiments, the T2R receptor antagonist peptide consists of the amino acid sequence AGDDAPRAVF (SEQ ID No:5) or ETSARHL (SEQ ID No:4).

In some embodiments, the T2R receptor antagonist peptide is a T2R4 or T2R14 receptor antagonist peptide.

According to another aspect of the invention, there is provided a composition comprising a bitter taste blocking peptide, said peptide consisting of an amino acid sequence selected from the group consisting of: TMTL (SEQ ID No:1); ETCL (SEQ ID No:2); SSMSSL (SEQ ID No:3); ETSARHL (SEQ ID No:4); AGDDAPRAVF (SEQ ID No:5); AAMY (SEQ ID No:6); VSSY (SEQ ID No:7); and AAYM (SEQ ID No:8); and a food product.

In some embodiments, the food product is a food product that has a bitter taste.

In some embodiments, the food product is a food product that has agonist activity for T2R receptors.

According to another aspect of the invention, there is provided a composition comprising a bitter taste blocking peptide, said peptide consisting of an amino acid sequence selected from the group consisting of: TMTL (SEQ ID No:1); ETCL (SEQ ID No:2); SSMSSL (SEQ ID No:3); ETSARHL (SEQ ID No:4); AGDDAPRAVF (SEQ ID No:5); AAMY (SEQ ID No:6); VSSY (SEQ ID No:7); and AAYM (SEQ ID No:8); and a pharmaceutical product.

In some embodiments, the pharmaceutical product is a pharmaceutical product that has a bitter taste.

In some embodiments, the pharmaceutical product has side-effects consistent with activation of a T2R receptor.

According to a further aspect of the invention, there is provided a pharmaceutical composition comprising a bitter taste blocking peptide, said peptide consisting of an amino acid sequence selected from the group consisting of: TMTL (SEQ ID No:1); ETCL (SEQ ID No:2); SSMSSL (SEQ ID No:3); ETSARHL (SEQ ID No:4); AGDDAPRAVF (SEQ ID No:5); AAMY (SEQ ID No:6); VSSY (SEQ ID No:7); and AAYM (SEQ ID No:8); and a suitable excipient for co-administration with a pharmaceutical product.

In some embodiments, the pharmaceutical product is a pharmaceutical product that has a bitter taste.

In some embodiments, the pharmaceutical product has side-effects consistent with activation of a T2R receptor.

According to another aspect of the invention, there is provided a method of treating a food product comprising applying to said food product an effective amount of a bitter taste blocking peptide, said peptide consisting of an amino acid sequence selected from the group consisting of: TMTL (SEQ ID No:1); ETCL (SEQ ID No:2); SSMSSL (SEQ ID No:3); ETSARHL (SEQ ID No:4); AGDDAPRAVF (SEQ ID No:5); AAMY (SEQ ID No:6); VSSY (SEQ ID No:7); and AAYM (SEQ ID No:8); and a food product.

In some embodiments, the food product is a food product that has associated therewith a bitter taste.

### BRIEF DESCRIPTION OF THE DRAWINGS

FIG. 1. Molecular weight distribution of peptides present in enzymatic beef protein hydrolysates based on elution volume from a calibrated Superdex12 10/300 GL gel permeation column.

FIG. 2. Estimated e-tongue bitter scores of individual beef protein hydrolysates and their ability to suppress quinine bitterness intensity. AH, alcalase hydrolysate; CH, chymotrypsin hydrolysate; PH, pepsin hydrolysate; TH, trypsin hydrolysate; TMH, thermoase hydrolysate; FH, flavourzyme hydrolysate. BCML (Nα,Nα-bis(carboxymethyl)-1-lysine) was used as a positive control. Bars with different letters have significantly different (p<0.05) mean values. Bars labelled with uppercase or lowercase letters represent hydrolysate only or hydrolysate+quinine treatments, respectively.

FIG. 3. Calcium mobilization in T2R4-expressing HEK 293T cell system after treatment with beef protein hydrolysates (5 mg/mL) and quinine (1 mM). AH, alcalase hydrolysate; CH, chymotrypsin hydrolysate; PH, pepsin hydrolysate; TH, trypsin hydrolysate; TMH, thermoase hydrolysate; FH, flavourzyme hydrolysate. Bars with different letters have significantly different (p<0.05) mean values. ΔRFU: changes in relative fluorescence unit (test cells-control cells)

FIG. 4. Calcium mobilization in T2R4-expressing HEK 293T cell system after treatment with quinine (1 mM), beef protein hydrolysate peptide fractions (5 mg/mL) or a quinine (1 mM) solution that contained 5 mg/ml protein hydrolysate peptide fractions from first round of RP-HPLC separation. AH-F, alcalase hydrolysate fractions; CH-F, chymotrypsin hydrolysate fractions. Bars with different letters have significantly different (p<0.05) mean values. Inset shows the RP-HPLC separation and fraction collection. Bars labelled with uppercase or lowercase letters represent hydrolysate only or hydrolysate+quinine treatments, respectively.

FIG. 5. Calcium mobilization in T2R4-expressing HEK 293T cell system after treatment with quinine (1 mM), beef protein hydrolysate peptide fractions (5 mg/mL) or a quinine (1 mM) solution that contained 5 mg/ml alcalase hydrolysate peptide fractions (AH-F) from second round of RP-HPLC separation. Bars with different letters have significantly different (p<0.05) mean values. Inset shows the RP-HPLC separation and fraction collection. Bars labelled with uppercase or lowercase letters represent hydrolysate only or hydrolysate+quinine treatments, respectively.

FIG. 6. Calcium mobilization in T2R4-expressing HEK 293T cell system after treatment with quinine (1 mM), beef protein hydrolysate peptide fractions (5 mg/mL) or a quinine (1 mM) solution that contained 5 mg/ml alcalase hydrolysate peptide fractions (CH-F) from second round of RP-HPLC separation. Bars with different letters have significantly different (p<0.05) mean values. Inset shows the RP-HPLC separation and fraction collection. Bars labelled with uppercase or lowercase letters represent hydrolysate only or hydrolysate+quinine treatments, respectively.

FIG. 7. Calcium mobilization in HEK 293T cells stably expressing T2R4 and treated with different peptides. A. The T2R4 expressing cells were treated with quinine (1 mM), synthesized peptides (1 mM) or a quinine solution that contained the synthesized peptides. The calcium responses of cells treated with buffer are used as control. Statistically significant values are shown by asterisk (*p<0.05) and (***p<0.001). B. Raw traces for calcium mobilization analysis showing decrease in calcium release upon stimulation with different peptides (1-8) with quinine. The arrows at 20 sec indicates the addition of the compounds. The changes in fluorescence by the calcium sensitive dye Fluo-4NW are measured as relative fluorescence unit (ΔRFU) on the Y-axis for a total of 180 seconds (X-axis) using a Flex Station 3 plate reader.

FIG. 8. Peptides and quinine competition calcium mobilization assay on T2R4. HEK 293 T cells stably expressing T2R4 were treated with 1 mM quinine and with increasing concentrations of peptides ETSARHL, AGDDAPRAVF and AAMY ranging from 0.015-1 mM. Changes in intracellular calcium were measured (ΔRFUs), and IC₅₀ values were calculated using Graph Pad Prism 4.0. Data were collected from two-three independent experiments carried out in triplicate.

FIG. 9. Peptides and agonist competition calcium mobilization assays on T2R14. HEK293T cells stably expressing T2R14 were treated with the agonist diphenhydramine (0.5 mM) and with increasing concentrations of Peptide 4 (ETSARHL) and Peptide 5 (AGDDAPRAVF) ranging from 0.015 mM-1 mM. Changes in intracellular calcium were measured (ΔRFUs), and IC50 values were calculated using GraphPad Prism 4.0. Both peptides show almost similar IC50 of 121±65 μM and 118±50 μM for T2R14 activated by diphenhydramine. Data were collected from a minimum of two independent experiments carried out in triplicate.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

The aim of this work was to determine the bitter taste-blocking ability of enzymatic beef protein hydrolysates and identified peptide sequences. Beef protein was hydrolyzed with each of six commercial enzymes (alcalase, chymotrypsin, trypsin, pepsin, flavourzyme, and thermoase). Electronic tongue measurements showed that the hydrolysates had significantly (p<0.05) lower bitter scores than quinine. Addition of the hydrolysates to quinine led to reduced bitterness intensity of quinine with trypsin and pepsin hydrolysates being the most effective. Addition of the hydrolysates to HEK293T cells that heterologously express one of the bitter taste receptors (T2R4) showed alcalase, thermoase, pepsin and trypsin hydrolysates as the most effective in reducing calcium mobilization.

After a typical protein hydrolysis, up to 300-500 peptides can be generated. The use of column fractionation techniques coupled with bioassay tests to separate and identify the most active sequences allowed for the bitter taste blockers to be isolated from the hundreds of peptides that were originally generated. This was accomplished by first separating the hydrolysates on a reverse-phase HPLC column with matrix consisting of carbon 12 (C-12) materials. The peptides bind to the column based on their non-polar (hydrophobic) character. By using a solvent gradient elution, peptides can be detached and eluted from the column starting from the least tightly bound to the most-tightly bound. In the first round, the peptides were collected every minute into separate tubes (fractions) and then evaluated for ability to antagonize T2R4 using a cell culture technique. The most active fraction was then passed through the HPLC column again (second round) but this time using a different solvent gradient to separate the peptides again into fractions, which were then tested for antagonism of T2R4. The most active fractions from the second round were then passed through a mass spectrometer, which separates the peptides based on individual weight (mass). Each mass (representing one peptide) was further passed through a second mass spectrometer chamber where the peptide is broken down one amino acid at a time (called daughter ions) to generate a second mass chromatogram. The daughter ion data were then analyzed by PEAK software which deconvolutes the information to yield the correct arrangement of amino acids on the peptide chain.

According to an aspect of the invention, there is provided a method of isolating and identifying a bitter taste blocker peptide from a protein hydrolysate comprising:

providing a quantity of a protein;

generating one or more hydrolysates by hydrolyzing the protein with a protease;

separating the one or more hydrolysates into fractions using a first peptide separation technique;

evaluating each respective one of the fractions for ability to antagonize a bitter taste receptor (T2R) in cell culture and selecting the most active fractions;

separating the most active fractions into sub-fractions using a second peptide separation technique;

evaluating each respective one of the sub-fractions for ability to antagonize a bitter taste receptor (T2R) in cell culture and selecting the most active sub-fractions; and

passing each respective one most active sub-fraction through a mass-spectrometer, thereby identifying a bitter taste blocker peptide.

The one or more hydrolysates may be prepared using one or more proteases. Suitable proteases will be readily apparent to one of skill in the art. However, examples of suitable proteases include but are by no means limited to: alcalase, thermoase, pepsin, trypsin, flavourzyme, chymotrypsin, protease S, protex 6L and protex 50FP.

As will be appreciated by one of skill in the art, the term “protein” as used herein does not necessarily refer to a preparation of a single isolated protein or peptide but may also refer to a preparation of proteins from animal or plant tissue comprising diverse proteins and/or peptides.

In some embodiments, the protein is a food-derived protein preparation and/or a protein preparation prepared from animal or plant tissue.

Previous reports suggested that peptides isolated from food proteins (cheese, soybean, casein etc.) only taste bitter and active the bitter taste receptors. Our discovery was the first to show that peptides isolated from food protein hydrolysates can in fact block bitter taste receptors. This was a paradigm shift in the field, in terms of identifying natural blockers for the bitter taste receptors. Accordingly, it is a sound prediction that other protein sources, for example, non-food protein sources, will contain bitter taste blocker peptides.

As will be appreciated by one of skill in the art, a “food protein” source refers to a substance consumed as part of a regular or normal diet.

According to an aspect of the invention, there is provided a method of isolating and identifying a bitter taste blocker peptide from a protein hydrolysate comprising:

providing a quantity of protein;

generating a first protein hydrolysate by hydrolyzing a first portion of the protein with a first protease;

generating a second protein hydrolysate by hydrolyzing a second portion of the protein with a second protein protease

generating a third protein hydrolysate by hydrolyzing a third portion of the protein with a third protease;

generating a fourth protein hydrolysate by hydrolyzing a fourth portion of the protein with a fourth protease;

separating each protein hydrolysate into fractions using a first peptide separation technique;

evaluating each respective one of the fractions for ability to antagonize a bitter taste receptor (T2R) in cell culture and selecting the most active fractions;

separating the most active fractions into sub-fractions using a second peptide separation technique;

evaluating each respective one of the sub-fractions for ability to antagonize a bitter taste receptor (T2R) in cell culture and selecting the most active sub-fractions; and

passing each respective one most active sub-fraction through a mass-spectrometer, thereby identifying a bitter taste blocker peptide.

In some embodiments, the first, second, third and fourth proteases are alcalase, thermoase, pepsin and trypsin.

In other embodiments, the method further comprises generating a fifth food protein hydrolysate by hydrolyzing a fifth portion of the food-derived protein with a fifth protease; and generating a sixth food protein hydrolysate by hydrolyzing a sixth portion of the food derived protein with a sixth protease. In these embodiments, the fifth and sixth proteases are flavourzyme and chymotrypsin.

In some embodiments of the invention, the fractions and sub-fractions are separated on a column, that is, are column-purified. In some embodiments, the column is an HPLC-column, for example, a reverse-phase HPLC column. As will be appreciated by one of skill in the art, there are a wide variety of separation techniques known in the art, depending for example on the matrix and/or solvent selected.

As discussed above, a key aspect of the invention is the use of different separation techniques for generating the fractions and sub-fractions.

As discussed herein, bitter taste receptors are expressed in different cell types in humans from brain cells to testis. As such, a wide variety of cells may be used for screening for bitter taste receptor blockers and such cell types will be readily apparent to one of skill in the art. In some embodiments, the bitter taste receptor is selected from T2R14, T2R4, T2R20, T2R10, T2R3, T2R5, and T2R7.

In other embodiments, the bitter taste receptor is selected from T2R14, T2R4, T2R20, T2R10, T2R3 and T2R5.

In other embodiments, the bitter taste receptor is selected from T2R14, T2R4, T2R20, T2R10 and T2R3.

In other embodiments, the bitter taste receptor is selected from T2R14, T2R4, T2R20 and T2R10.

In other embodiments, the bitter taste receptor is selected from T2R14, T2R4 and T2R20.

In other embodiments, the bitter taste receptor is selected from T2R14 and T2R4.

In other embodiments, the bitter taste receptor is T2R14.

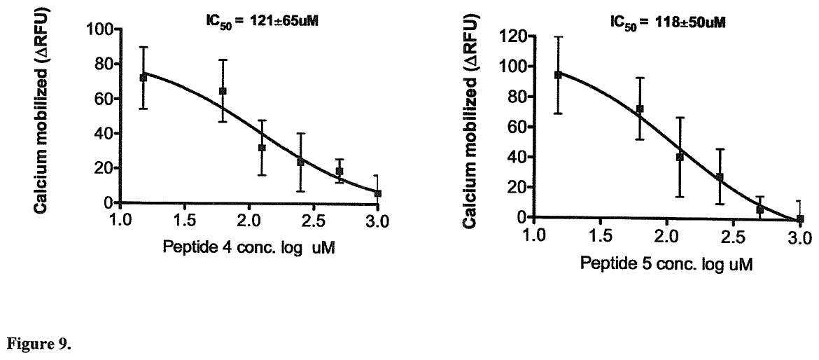

Eight peptides that were identified from the alcalase and chymotrypsin hydrolysates also suppressed quinine-dependent calcium release from T2R4: 1. TMTL (SEQ ID No:1), 2. ETCL (SEQ ID No:2), 3. SSMSSL (SEQ ID No:3), 4. ETSARHL (SEQ ID No:4), 5. AGDDAPRAVF (SEQ ID No:5), 6. AAMY (SEQ ID No:6), 7. VSSY (SEQ ID No:7), and 8. AAYM (SEQ ID No:8), with AGDDAPRAVF (SEQ ID No:5) and ETSARHL (SEQ ID No:4) being the most effective, as discussed below. The result showing 8 different peptide sequences was surprising since not many natural peptides have been isolated in this way as bitter taste blockers. We conclude that short peptide lengths or the presence of multiple serine residues may not be desirable structural requirements for blocking quinine-dependent T2R4 activation. Next, we tested the ability of the most effective peptides, ETSARHL (SEQ ID No:4), and AGDDAPRAVF (SEQ ID No:5) to block another T2R, T2R14. The results suggest that both peptides are effective in blocking diphenhydramine-dependent T2R14 activation (FIG. 9).

According to an aspect of the invention, there is provided a bitter taste blocking peptide, said peptide consisting of an amino acid sequence selected from the group consisting of: TMTL (SEQ ID No:1); ETCL (SEQ ID No:2); SSMSSL (SEQ ID No:3); ETSARHL (SEQ ID No:4); AGDDAPRAVF (SEQ ID No:5); AAMY (SEQ ID No:6); VSSY (SEQ ID No:7); and AAYM (SEQ ID No:8).

In some embodiments, the bitter taste blocking peptide consists of the amino acid sequence AGDDAPRAVF (SEQ ID No:5) or ETSARHL (SEQ ID No:4).

In some embodiments, the bitter taste blocking peptide is a T2R4 or T2R14 receptor antagonist.

According to an aspect of the invention, there is provided a T2R4 or T2R14 receptor antagonist peptide, said peptide consisting of an amino acid sequence selected from the group consisting of: TMTL (SEQ ID No:1); ETCL (SEQ ID No:2); SSMSSL (SEQ ID No:3); ETSARHL (SEQ ID No:4); AGDDAPRAVF (SEQ ID No:5); AAMY (SEQ ID No:6); VSSY (SEQ ID No:7); and AAYM (SEQ ID No:8).

In some embodiments, the T2R4 or T2R14 receptor antagonist peptide consists of the amino acid sequence AGDDAPRAVF (SEQ ID No:5) or ETSARHL (SEQ ID No:4).

As will be appreciated by one of skill in the art, the peptides may be prepared and/or isolated by any suitable means known in the art.

In some embodiments, the peptides are arranged for addition to finished food products, including cooked or otherwise prepared food items, beverages, and the like.

In some embodiments, the peptides may be freeze-dried and packaged to be applied as a powder to a food product such as for example a prepared food or beverage This work has revealed the potential to use beef protein hydrolysates and derived peptides as bitter taste blockers, specifically against T2R4 and T2R14. Recent studies have revealed that T2Rs are also expressed in the gastrointestinal tract, enteroendocrine STC-1 cells, respiratory system, male reproductive system, central nervous system and several tissues.⁴⁹⁻⁵¹ This development suggests that T2Rs may possess more potential important physiological functions other than bitter taste sensation. For example, most drugs have bitter taste, which may be responsible for some of the observed off-target (or negative) effects since these drugs can activate T2Rs in non-target parts of the body. Therefore, in addition to enhancing eating quality of foods, potent bitter taste-suppressing peptides may find additional uses as agents to reduce off-target effects of certain drugs. Moreover, since the mechanism of signal transduction of T2Rs in different cell types are not completely understood, peptides with inhibitory ability may be used to explore the signaling pathways in different cell types. Since only one T2R was studied in this work, future research works will determine the inhibitory effects of these food protein-derived hydrolysates and peptides against multiple T2Rs.

According to another aspect of the invention, there is provided a composition comprising a bitter taste blocking peptide, said peptide consisting of an amino acid sequence selected from the group consisting of: TMTL (SEQ ID No:1); ETCL (SEQ ID No:2); SSMSSL (SEQ ID No:3); ETSARHL (SEQ ID No:4); AGDDAPRAVF (SEQ ID No:5); AAMY (SEQ ID No:6); VSSY (SEQ ID No:7); and AAYM (SEQ ID No:8); and a food product.

According to another aspect of the invention, there is provided a composition comprising a bitter taste blocking peptide, said peptide consisting of an amino acid sequence selected from the group consisting of: TMTL (SEQ ID No:1); ETCL (SEQ ID No:2); SSMSSL (SEQ ID No:3); ETSARHL (SEQ ID No:4); AGDDAPRAVF (SEQ ID No:5); AAMY (SEQ ID No:6); VSSY (SEQ ID No:7); and AAYM (SEQ ID No:8); and a pharmaceutical product.

In some embodiments, the pharmaceutical product is a pharmaceutical product that has a bitter taste or that has side-effects consistent with activation of a T2R, for example, T2R4 or T2R14.

As will be appreciated by one of skill in the art, the bitter taste blocking peptide may be co-formulated with the pharmaceutical product, for example, a medicament.

According to another aspect of the invention, there is provided a pharmaceutical composition comprising a bitter taste blocking peptide, said peptide consisting of an amino acid sequence selected from the group consisting of: TMTL (SEQ ID No:1); ETCL (SEQ ID No:2); SSMSSL (SEQ ID No:3); ETSARHL (SEQ ID No:4); AGDDAPRAVF (SEQ ID No:5); AAMY (SEQ ID No:6); VSSY (SEQ ID No:7); and AAYM (SEQ ID No:8); and a suitable excipient for co-administration with a pharmaceutical product.

In some embodiments, the pharmaceutical product is a pharmaceutical product that has a bitter taste or that has side-effects consistent with activation of a T2R, for example, T2R4 or T2R14.

According to another aspect of the invention, there is provided a method of treating a food product comprising applying to said food product an effective amount of a bitter taste blocking peptide, said peptide consisting of an amino acid sequence selected from the group consisting of: TMTL (SEQ ID No:1); ETCL (SEQ ID No:2); SSMSSL (SEQ ID No:3); ETSARHL (SEQ ID No:4); AGDDAPRAVF (SEQ ID No:5); AAMY (SEQ ID No:6); VSSY (SEQ ID No:7); and AAYM (SEQ ID No:8); and a food product.

In some embodiments, the food product is a food product that has associated therewith a bitter taste.

In yet other embodiments, the peptides of the invention may be formulated as a powder for addition to a beverage such as water or formulated as an oral care product or oral hygiene product for modifying or blocking bitter taste.

As will be appreciated by one of skill in the art, “an effective amount” will depend on a variety of factors, for example, the bitterness of the food product and the preference of the individual who will consume the food product.

The invention will now be further explained and elucidated by way of examples; however, the invention is not necessarily limited to the examples.

### Example 1: Degree of Hydrolysis

As shown in Table 1, the degree of hydrolysis (DH) of the protein hydrolysates was enzyme-dependent with the highest values for the microbial enzymes alcalase and flavourzyme. The high DH for flavourzyme may have been due to the presence of endoproteases and exoproteases,³² which could have enhanced the rate of proteolysis. The results are similar to those previously reported for bovine plasma proteins where the flavourzyme hydrolysates had higher DH values than the alcalase hydrolysates.³³ Similarly, a slightly higher DH for a peanut hydrolysate produced from flavourzyme when compared to that of alcalase after hydrolysis for 4 h was suggested. However, the results are different from those reported for tilapia muscle protein hydrolysis where the flavourzyme produced lower DH than alcalase.²⁴ An interesting outcome is that the intestinal enzymes (trypsin and chymotrypsin) digested the meat proteins more efficiently and produced hydrolysates with higher DH than the stomach enzyme, pepsin.

The beef proteins were hydrolyzed separately using each of these enzymes. The cleavage point for each enzyme depends on the type of the two amino acids that are joined by a particular peptide bond. Therefore, because the specificity of each enzyme for the type of amino acids involved in the peptide bond formation is different, the length and amino acid sequence of liberated peptides will differ for each enzyme.

### Example 2—Amino Acid Composition

In comparison to the defatted beef protein, significant changes in some of the amino acids were observed after enzymatic hydrolysis (Table 2). Glutamic acid+glutamine (Glx) were the most abundant in the beef but there was no significant change in content after protein hydrolysis. In contrast, the contents of aspartic acid+asparagine (Asx) and arginine were significantly (p<0.05) enhanced in the protein hydrolysates. However, the protein hydrolysates had significantly reduced levels of cysteine, methionine and tryptophan. Kohl et al.³⁴ had previously shown that tryptophan is a T2R4 agonist and potentiator of bitterness intensity. Therefore, protein hydrolysates with lower tryptophan levels may provide a better source of weakly-acting T2R4 peptide agonists or even antagonists when compared to those with higher levels. With respect to amino acid groups, there were overall significant (p<0.05) reductions in hydrophobic amino acids, aromatic amino acids and sulfur-containing amino acids after the enzymatic protein hydrolysis. But positively-charged amino acids were significantly (p<0.05) higher in the protein hydrolysates when compared to the defatted beef protein. A previous work has shown that the peptide's amino acid side groups may be more important than the amino and carbonyl groups during T2R4 activation.³⁴ Therefore, the results reflect the different proteolytic specificities of the proteases used in this work, which provided a wide variation of peptides (different side groups) that could function as T2R4 antagonists.

### Example 3: Gel-Permeation Chromatography

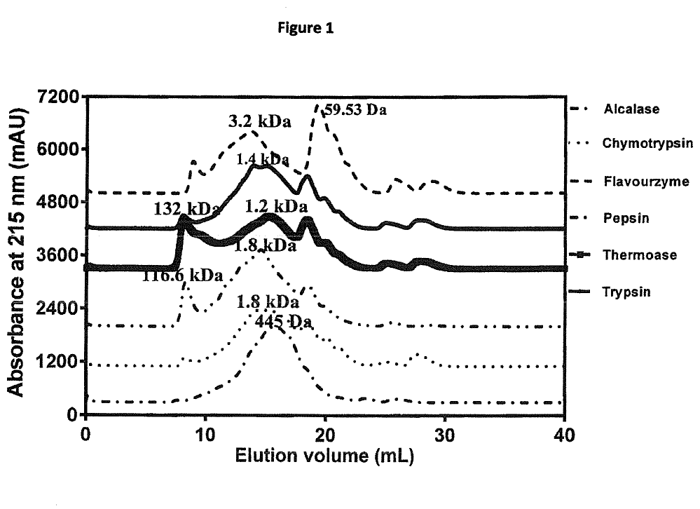

Peptide size distribution indicates the presence of mostly peptides with the main peaks between 0.445-3.2 kDa (FIG. 1). Alcalase hydrolysate (AH) had the most uniform peptide distribution followed by chymotrypsin hydrolysate (CH) while the remaining hydrolysates had big peptide peaks with estimated MW of 116-132 kDa. Alcalase is an endopeptidase with a very high degree of proteolysis, which may have contributed to the almost normal distribution of low molecular peptides. However, unlike the other hydrolysates, the flavourzyme hydrolysate (FH) had a distinct big peak with ˜60 Da estimated MW, which most likely represents amino acids due to the presence of exopeptidase activity. Peptide size and amino acid composition play an important role in taste. This is because previous studies have reported that peptides of 0.36-2.10 kDa were primary contributors to bitterness of protein hydrolysates, because smaller peptides failed to achieve the particular conformation required for binding to taste receptors.^{35,36} These results suggest that peptide sizes identified from this work fall within the 0.36-2.10 kDa range and can interact with bitter taste receptors.

### Example 4: Prediction of Bitter Score from E-Tongue

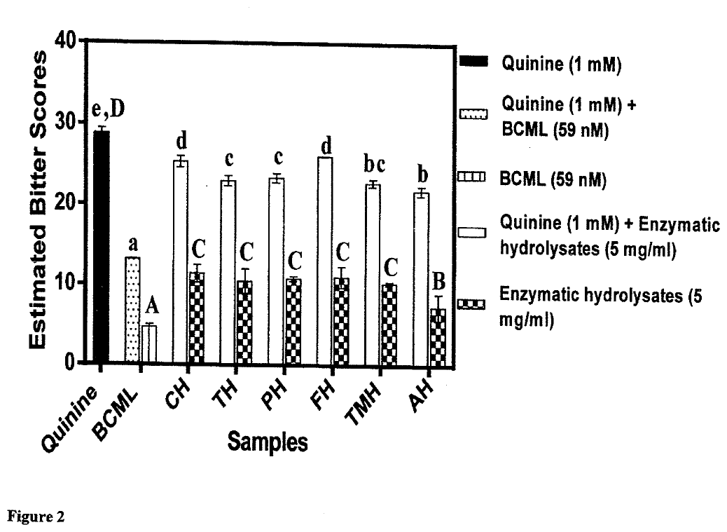

Electronic-tongues have been widely used to detect bitter taste of samples, but also can determine the suppression ability of bitter taste modifiers, such as high potency sweeteners suppressing bitter taste of quinine hydrochloride, and acesulfame K and citric acid suppressing the bitter taste of epinephrine.^{37,33} Quinine can activate multiple T2Rs³⁹ and therefore is a suitable compound to test for antagonists or bitter taste suppressors. FIG. 2 shows e-tongue bitterness scores for individual beef protein hydrolysates and their combinations with quinine.

Quinine had the strongest bitterness intensity while the inverse agonist for T2R4 (BCML) had the least (p<0.05). Among the hydrolysates, AH had the least bitterness intensity (p<0.05) while there were no significant differences between the remaining five hydrolysates. The lower bitterness intensity of the AH may be attributed to two main factors. First, is the higher DH (compared to the other hydrolysates), which could have enhanced further proteolysis to split very bitter and larger peptides which would in turn produce smaller peptides with reduced bitter-taste. This is consistent with the lower molecular weight profile (˜445 Da) of the AH peptides when compared to the other hydrolysates (FIG. 1). The possibility of using extensive proteolysis to decrease protein hydrolysate bitterness has also been discussed.⁴⁰ Second, the sum of hydrophobic amino acids (HAA) and positively-charged amino acids (PCAA) has been reported to be important for enhancing bitterness intensity of peptides.⁴¹ AH and FH had the lowest sum of HAA+PCAA but it can be concluded that FH has less content of peptides and more free amino acids due to the exopeptidase activity in flavourzyme. Therefore, the lower bitterness intensity of AH may also be due to the reduced contents of PCAA and HAA. The results are consistent with previous works that have reported that enzymatic food protein hydrolysates usually have bitter taste (Humiski and Aluko).^{36,42,43} In the presence of BCML and each of the protein hydrolysates, bitterness intensity of quinine was significantly (p<0.05) decreased. BCML produced the most decrease in quinine bitterness intensity followed by TH and PH. The structural basis for the bitter taste blocking efficiency of the hydrolysates is difficult to determine because of the presence of a large pool of peptides. However, it is pertinent to note that the TH had the lowest content of HAA while PH had the least content of negatively-charged amino acids.

### Example 5: Inhibition of Quinine-Dependent T2R4 Activation by Protein Hydrolysates

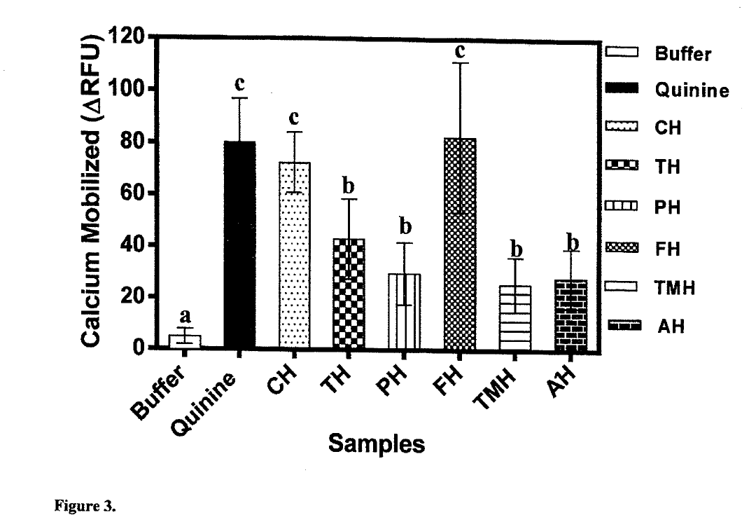

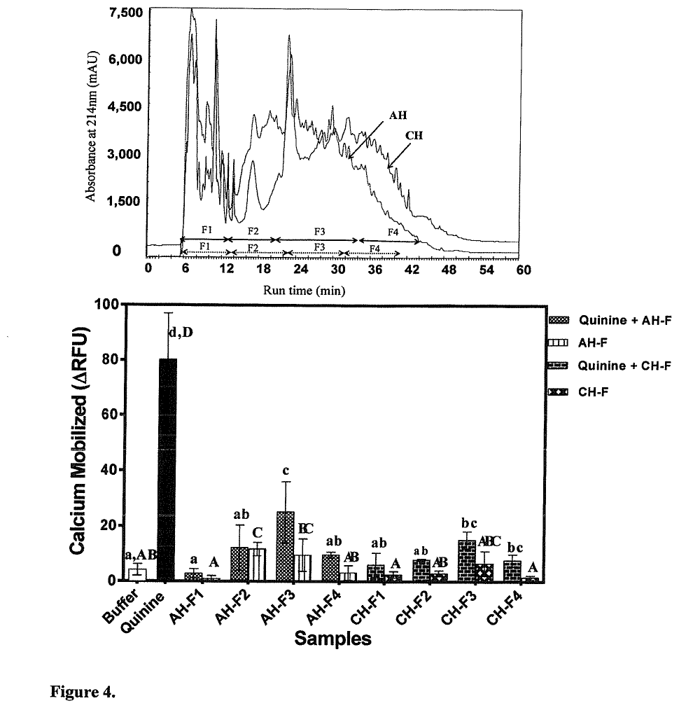

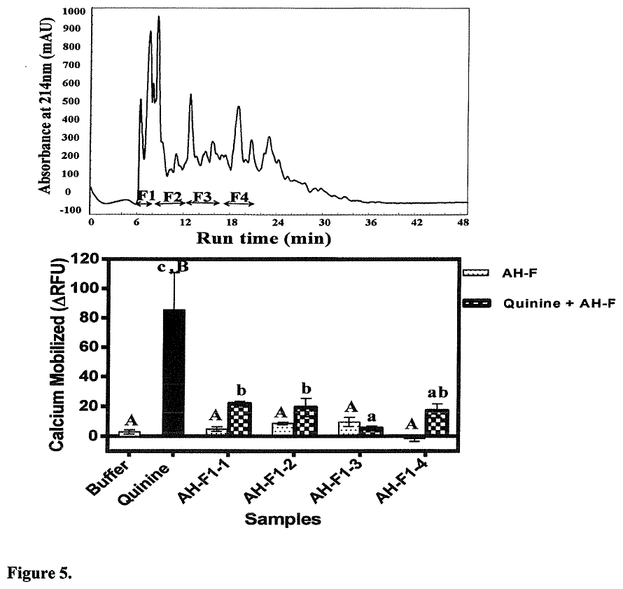

In previous studies that examined small molecular weight bitter taste modifiers, T2Rs were heterologously expressed in HEK293T cells and an intracellular calcium mobilization assay was applied to measure the bitterness inhibitory ability of the compounds.^{5,6,7} In this assay, high intracellular calcium mobilization means that the T2Rs are activated more intensely while lower calcium releases suggest weak activation. Results from the calcium mobilization assay indicate highest activity for quinine, CH and FH (FIG. 3). In contrast, TH, PH, TMH and AH were significantly less effective in inducing intracellular calcium mobilization in T2R4 expressing cells. The low calcium mobilization ability of AH is consistent with the lowest bitterness intensity among the hydrolysates as determined by e-tongue. However, the calcium mobilization ability of the remaining five hydrolysates was not related to the e-tongue data. The uniform peptide size distribution in AH when compared to the other hydrolysates with non-uniform size distribution may have contributed to this discrepancy. The results suggest that high molecular weight peptides (more abundant in FH, CH, TH, PH and TMH) behave differently from smaller molecular weight peptides (more abundant in AH) with respect to electrochemical signaling properties in the e-tongue and biological interactions with the T2R4. Moreover, previous reports have suggested the e-tongue instrument appears to have difficulty in sensing organic bitter taste substances, such as amino acids and peptides; therefore, a wider variation in peptide size could have exacerbated this deficiency.^{44,45} Because the e-tongue responses varied from those of the calcium mobilization assay, the least effective (AH) and most effective (CH) in causing calcium release from T2R4 cells were chosen for peptide identification through RP-HPLC separation to obtain 4 fractions each (FIG. 4).

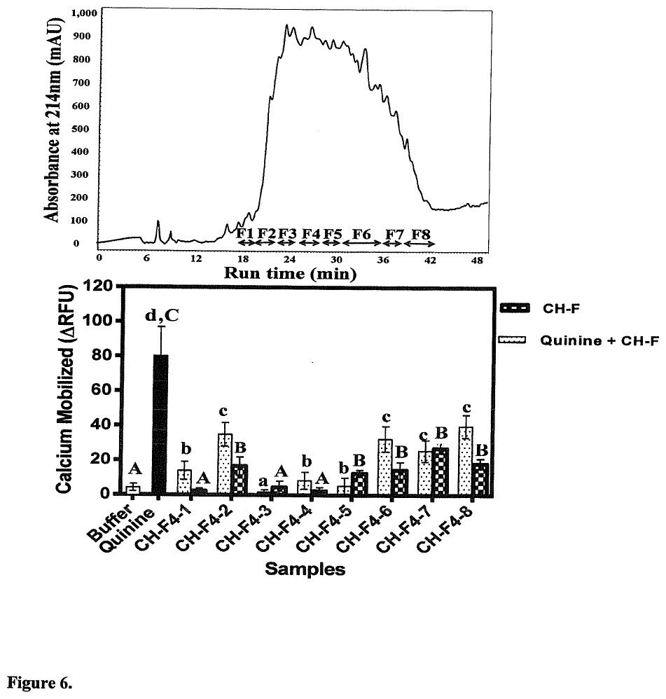

The AH-F1 was the most effective in blocking quinine-dependent calcium release in the T2R4 expressing HEK293T cells while the four CH fractions behaved similarly to each other. However, CH-F4 was chosen for further peptide fractionation due to higher abundance when compared to CH-F1, CH-F2 and CH-F3. AH-F1 separation on the RP-HPLC column also led to 4 isolated fractions with the AH-F3-3 producing the most significant (p<0.05) blockage of quinine-dependent calcium release from T2R4 expressing HEK293T cells (FIG. 5). In contrast, RP-HPLC separation of CH-F4 yielded 8 fractions, all which produced significant (p<0.05) reductions in calcium release when added to quinine (FIG. 6). However, CH-F4-1, CH-F4-3, CH-F3-4 and CH-F3-5 had the most reductions in calcium release. Based on absolute values of the decrease in calcium release, AH-F1-3, CH-F4-3 and CH-F4-5 were chosen for peptide identification and amino acid sequencing.

### Example 6: Inhibition of Quinine-Dependent T2R4 Activation by Synthesized Peptides

Four peptides were identified from AH-F1-3, one from CH-F4-3 and three from CH-F4-5 with sequences shown in Table 3. Threonine, serine, methionine, leucine, and alanine were the most common amino acids in the peptides. With the exception of AAMY and AAYM, the ratio of hydrophobic amino acids in each of the identified peptides was less than 50%, which suggest that hydrophilic characteristics may have contributed to the bitter taste-suppressing ability of these peptides. However, it has been suggested that hydrophobic properties can enhance peptide entry into target organs through cell membrane lipid bilayer,⁴⁶ which contributes to enhanced bioactivity inside the cells. Besides, some hydrophobic compounds, such as D-Tryptophan benzyl ester and N,N-Dibenzyl-L-serine methyl ester, were reported to have high predicted antagonistic binding affinity to T2R4,⁵ implying that it is possible for hydrophobic substances to inhibit bitter taste receptors. Thus, peptides with high contents of hydrophobic amino acids may also have ability to suppress bitterness.

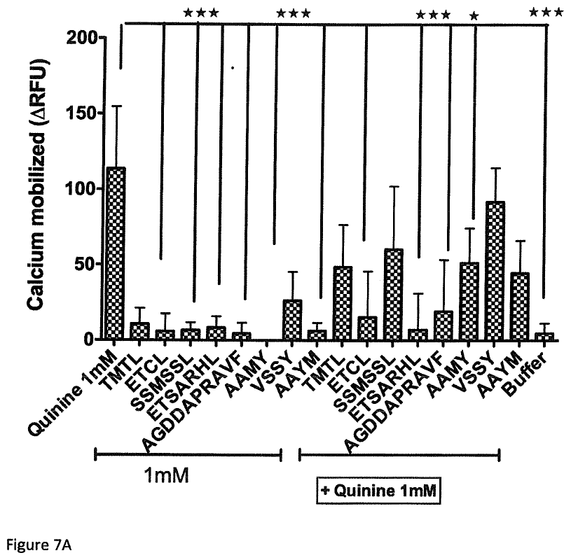

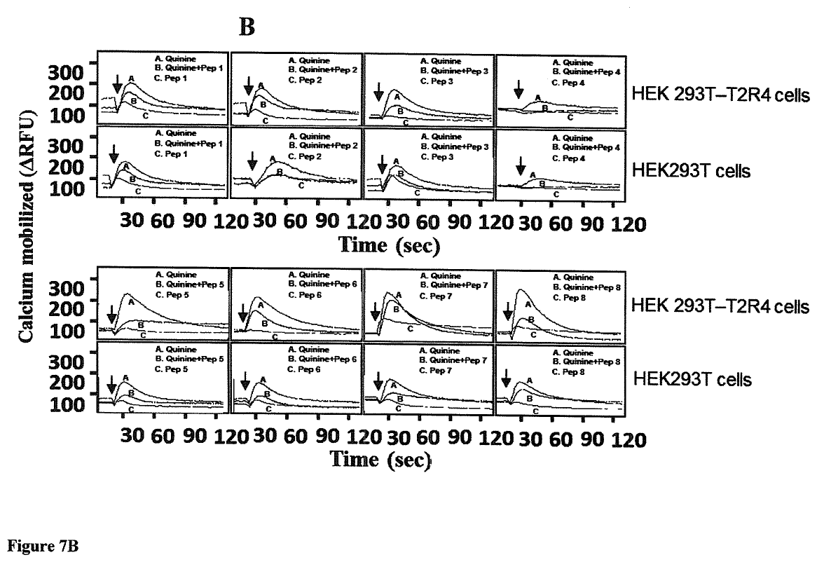

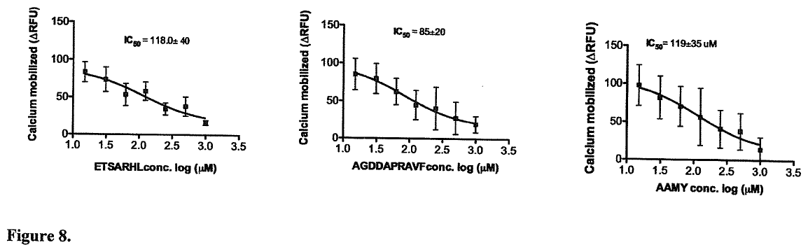

All peptides showed significantly (p<0.05) lower calcium mobilization than quinine (FIG. 7). Peptides ETSARHL (SEQ ID No:4), AGDDAPRAVF (SEQ ID No:5) and AAMY (SEQ ID No:6) showed less calcium mobilization (ΔRFU) upon co-incubation with quinine in HEK 293T cells expressing T2R4 indicating that these three peptides may have triggered calcium mobilization through T2R dependent pathway. Next, competition assays on T2R4 were pursued using these three peptides to obtain their inhibitory concentrations (IC₅₀). Result showed that these peptides inhibited quinine response in a concentration dependent manner, with AGDDAPRAVF (SEQ ID No:5) showing a lower IC₅₀ of 85 μM among the three peptides analyzed (FIG. 8).

It has been observed in several soybean protein-derived peptides that leucine residue at C-terminal was responsible for bitterness; treatment with a carboxypeptidase led to a marked reduction in bitterness intensity.^{47,48} Hence the peptides AGDDAPRAVF (SEQ ID No:5), AAMY (SEQ ID No:6), VSSY (SEQ ID No:7), and AAYM (SEQ ID No:8) should have a less bitter taste compared to other identified peptides with a leucine residue at C-terminal. But the results obtained in this work do not agree with such hypothesis because the leucine-containing peptides had similar or even lower calcium release ability than peptides that did not contain leucine (FIG. 7). Addition of each peptide to quinine led to significant reductions (except VSSY (SEQ ID No:7)) in calcium release from the T2R4 cells with AGDDAPRAVF (SEQ ID No:5) being the most effective blocker. The two peptides with multiple numbers of serine residues were not very effective, which suggests that this amino acid may not be an important structural requirement for bitter taste-blocking peptides. However, the number of peptides studied in this work is too small to estimate structure-function properties. But it is important to point out that the two most active suppressors of quinine bitter taste are also the longest peptide chains, which could indicate an importance for the number of amino acids.

### Example 7: Inhibition of Agonist-Dependent T2R14 Activation by Synthesized Peptides

To generalize the applicability of these peptides in blocking other T2Rs, we choose T2R14 as our next target. T2R14 is a bitter receptor highly expressed in humans, hence a valid choice to test the antagonism of the two peptides, ETSARHL (SEQ ID No:4) and AGDDAPRAVF (SEQ ID No:5). Our competition calcium mobilization assays suggest that both peptides also block diphenhydramine-dependent T2R14 activation, with an IC₅₀ of around 100 μM (FIG. 9).

### Materials and Methods

Materials. Ground beef was purchased from a local market (Safeway, Winnipeg, Manitoba, Canada). Chymotrypsin® (from bovine pancreas, EC 3.4.21.1), Trypsin® (from porcine pancreas, EC 3.4.21.4), Pepsin® (from porcine gastric mucosa, EC 3.4.23.1), Alcalase® (from fermentation of Bacillus licheniformis, EC 3.4.21.62), and Flavourzyme® (from Aspergillus oryzae, EC 232.752.2) were all purchased from Sigma-Aldrich (St. Louis, Mo., USA). Thermoase® (from Bacillus stearothermophilus, EC 3.4.24.27) was a product of Amano Enzymes Inc. (Nagoya, Japan). Electronic tongue instrument diagnostic solutions including hydrochloride (0.1 M HCl), sodium chloride (0.1 M NaOH) and monosodium glutamate (0.1 M MSG) as well as the calibration solution (1 M HCl) were purchased from Alpha M.O.S (Toulouse, France). Known bitter score substances such as acetaminophen, caffeine monohydrate, quinine hydrochloride (QHCI), leporamide hydrochloraide and femotidine were purchased from MP Biomedicals (Solon, Ohio, USA). Quinine and BCML (Nα,Nα-bis(carboxymethyl)-I-lysine) was from Sigma Aldrich (Oakville, ON, Canada).

Preparation of beef protein hydrolysates (BPHs). Raw ground beef (approximately 250 g) was evenly packed into aluminum foil plates, frozen at −20° C. for 24 h and then freeze-dried. The freeze-dried beef was blended thereafter in a Waring blender to fine powder and defatted repeatedly by mixing 100 g with 1 L food grade acetone. The mixture was continually stirred for 3 h in fume hood and decanted manually followed by two consecutive extractions of the residues. The defatted beef (DB) was placed in aluminum foil plates and air-dried overnight in the fume hood at room temperature. The DB was milled in the Waring blender into fine powder and stored at −20° C. DB was then mixed with water to prepare 5% (w/v) a suspension followed by addition of an enzyme (1%, w/w, protein weight basis) to initiate protein hydrolysis. The hydrolysis conditions (temperature and pH) of each enzyme were based on manufacturers' instructions and literature information.²¹⁻²³ For alcalase hydrolysis, the DB suspension was heated to 55° C. and adjusted to pH 8.0 using 2 M NaOH. The DB suspensions for trypsin, chymotrypsin and thermoase hydrolysis were first heated to 37° C. and then adjusted to pH 8.0. For flavourzyme and pepsin hydrolysis, the DB suspension was heated to 50° C. and 37° C. followed by adjustment to pH 6.5 and 2.0, respectively. Each mixture was stirred continuously for 4 h and the reaction terminated by heating at 95° C. for 15 min. The reaction mixtures were thereafter centrifuged (3,270 g at 4° C.) for 30 min and the resulting supernatants freeze-dried as the BPHs and stored at −20° C.

Determination of degree of hydrolysis. The DH of various BPHs was determined by the O-phthalic aldehyde (OPA) method, which was based on previous reports.^{24,25} The OPA reagent, which was prepared fresh daily contained 6 mM OPA dissolved in 95% methanol and 5.7 mM DL-dithiothreitol in 0.1 M sodium tetraborate decahydrate with 2% (w/v) SDS. N-Gly-Gly glycine solution was prepared as standard in 8 serial concentrations (0.05-0.4 mg/mL) while DB and BPHs were prepared in distilled water at 0.25 mg/mL. Ten μL of the standard solutions, DB or BPH were pipetted into microplate wells followed by addition of 200 μL of OPA reagent. Absorbance of the standard and samples were then read at 340 nm and 37° C. in a Synergy H4 multi-mode plate reader (Biotek Instruments, Winooski, Vt., USA). The total amino groups in the DB were determined using a sample that has been subjected to 6 M HCl hydrolysis at 110° C. for 24 h. The DH was calculated by the following equation:

% DH=[((NH₂)_{BPH})−(NH₂)_{DB}/((NH₂)_{Total}−(NH₂)_{DB})]*100

(NH₂)_{BPH}: Content of free amino groups in BPH
(NH₂)_{DB}: Content of free amino groups in DB
(NH₂)_{Total}: Content of free amino groups in acid hydrolyzed DB

Analysis of amino acid composition. The amino acid profiles of DB and BPH were determined according the method of Bidlingmeyer,²⁶ using an HPLC system to analyze amino acid composition of samples that have been hydrolyzed with 6 M HCl for 24 h. The contents of cysteine and methionine were measured after performic acid oxidation²⁷ while tryptophan content was determined after alkaline hydrolysis.²⁸

Estimation of molecular weight distribution. Molecular weight distribution of BPH was determined based on the method described by He et al.,²⁹ using an AKTA FPLC system (GE Healthcare, Montreal, PQ) equipped with a Superdex Peptide12 10/300 GL column 154 (10×300 mm) and UV detector (A=214 nm). The column was calibrated using the following standard proteins and an amino acid: cytochrome C (12,384 Da); aprotinin (6,512 Da); vitamin, 855 Da); and glycine (75 Da). A 100 μL aliquot of the 5 mg/mL BPH sample (dissolved in 50 mM phosphate buffer, pH 7.0 containing 0.15 M NaCl) was loaded onto the column and elution performed at room temperature using the phosphate buffer at a flow rate of 0.5 mL/min. The molecular weights (MW) of peptides in samples were estimated from a linear plot of log MW versus elution volume of standards.

Estimation of bitter scores by electronic-tongue (e-tongue). Each BPH was dissolved in distilled water to give 0.5, 1.0, 2.5, 5.0, and 10.0 mg/mL concentrations followed by filtration first through a 0.45 μm filter disc and then a 0.2 μm filter. Bitter scores of filtered BPH solutions (20 mL) were evaluated using the Astree II E-tongue system (Alpha M.O.S., Toulouse, France). This system is a completely automated taste analyzer equipped with seven sensors, BD, EB, JA, JG, KA, OA, and JE, based on the ChemFET technology (Chemical modified Field Effect Transistor) to analyze liquid samples. Firstly, 0.01 M HCl was used to condition and calibrate sensors and the reference electrode repeatedly until stable signals were obtained for all seven sensors with minimal or no noise and drift. Secondly, diagnostic procedure was performed repeatedly using 0.1 M HCl, NaOH, and MSG to ensure the sensors can identify distinctive tastes, until the discrimination index achieved at least 0.94 on a principle component analysis (PCA) map. Thereafter, the PLS bitterness standard model was constructed using several bitter taste compounds with known bitter taste scores determined from human panelists, including 0.24 mM and 2.36 mM caffeine, 0.03 mM and 0.12 mM quinine, 0.44 mM and 0.88 mM prednisolone, as well as 3.31 mM and 19.85 mM paracetamol. Validation of the PLS model was achieved with 0.002 mM and 0.01 mM loperamide followed by 0.06 mM and 0.15 mM famotidine according to the manufacturer's instructions.³⁰ A series of BPH concentration (0.5 mg/mL, 1 mg/mL, 2.5 mg/mL, 5 mg/mL, and 10 mg/mL) were prepared, filtered as indicated above and then used to determine the e-Tongue threshold. The 5 mg/mL sample was determined as the maximum strength useful to evaluate BPH bitterness intensity and this concentration was subsequently used to obtain bitter scores. In order to evaluate the bitterness suppressing ability of protein hydrolysates, the T2R4 agonist quinine and BPH solutions were mixed to obtain 1 mM and 5 mg/mL concentrations, respectively. As positive control, the known T2R4 bitter taste blocker Nα,Nα-bis(carboxymethyl)-I-lysine (BCML) with an IC₅₀ of 59 nM for quinine were mixed together to obtain 59 nM (BCML) and 1 mM (Quinine) final concentrations, respectively. For all samples, triplicate analysis of each solution was performed.

Determination of cellular calcium mobilization. Determination of the potential bitter taste-activating or blocking activity of BPH was carried out by measuring intracellular Ca²⁺ mobilization using Fluo-4 NW calcium assay kit as previously described.³ Stable transfected HEK293 cells expressing T2R4 and G-alpha 16/44 or HEK293T cells expressing only G-alpha 16/44 were used as experimental and negative control group, respectively. Around 1×10⁵ cells/well plated in the 96-well BD-falcon biolux plate and then incubated at 37° C. in a CO₂ incubator for 16 h. After incubation, the culture medium was substituted (for 40 min at 37° C. in the CO₂ incubator followed by 30 min incubation at room temperature) with Fluo-4 NW dye solution, which contained lyophilized dye in 10 mL of assay buffer (1× Hanks' balanced salt solution, 20 mM HEPES) and 100 μL 2.5 mM probenecid added to prevent dye leakage from cytosol. Calcium mobilization was measured in terms of relative fluorescence units (RFUs) using a Flexstation-3 microplate reader (Molecular Devices, CA, USA) at 525 nm, following 494 nm excitation. Based on the e-tongue data, BPH at 5 mg/mL, BCML at 59 nM and agonist quinine at 1 mM were used individually or in combination with peptides to determine activation of T2R4. BPH or BCML was then mixed with quinine (1 mM final concentration) to obtain 5 mg/mL or 59 nM, respectively and used to determine inactivation of T2R4. The basal intracellular calcium levels were measured for the first 20 s followed by addition of appropriate concentration of ligands for another 120 s. To get the absolute RFUs, the basal RFU before adding the ligand, which was labeled minimum value (Min) was deducted from the maximum RFU (Max) obtained after stimulating with the ligand (absolute RFUs=Max−Min). Next, the signals from the negative control group cells were deducted from the observed signal of experimental group cells to give ΔRFUs. Data were collected from two independent experiments, each done in triplicate. PRISM software version 4.03 (GraphPad Software, San Diego) was used for data analysis.

Separation of BPH by reversed-phase high-performance liquid chromatography (RP-HPLC). Alcalase hydrolysate (AH) and chymotrypsin hydrolysate (CH), the two most active bitter taste blockers were subjected to RP-HPLC separation on a 21×250 mm C12 preparative column (Phenomenex Inc., Torrance, Calif., USA) attached to a Varian 940-LC system (Agilent Technologies, Santa Clara, Calif., USA) according to the method of Girgih et al.³¹ Briefly, freeze-dried AH or CH was dissolved in double distilled water that contained 0.1% trifluoroacetic acid (TFA) as buffer A to give 100 mg/mL. After sequential filtration through 0.45 μm and 0.2 μm filters, 4 mL of the sample solution was injected onto the C12 column. Fractions were eluted from the column at a flow rate of 10 mL/min using a linear gradient of 0-100% buffer B (methanol containing 0.1% TFA) over 60 min. Peptide elution was monitored at 214 nm, eluted peptides were collected using an automated fraction collector every 1 min and pooled into four fractions according to elution time. In this study, according to the distribution of peaks of chromatograms, 4 fractions each were collected from AH (AH-F1, AH-F2, AH-F3, AH-F4) and CH (CH-F1, CH-F2, CH-F3, CH-F4). The solvent in the pooled fractions was evaporated using a vacuum rotary evaporator maintained at a temperature range between 35 and 45° C. and thereafter the aqueous residue was freeze-dried. Fractionated peptides were analyzed for calcium mobilization ability using the cell culture protocols described above. The most active fractions (AH-F1 and CH-F4) were each subjected to a second round of RP-HPLC separation using peptide load (400 mg), C12 preparative column, elution buffers, flow rate and detection wavelength as used in the first round of separation. Elution was carried out with a linear gradient of 0-35% buffer B in buffer A over 49 min. Eluted peptides were collected using an automated fraction collector every 1 min and pooled into 4 AH-F1 fractions and 8 CH-F4 fractions. The solvent in each fraction was removed under vacuum in a rotary evaporator and the aqueous residues freeze-dried and used for calcium mobilization experiments as described above.

Peptide identification and sequencing. The most active fractions (AH-F1-3, CH-F4-3, CH-F4-5) against T2R activation (calcium mobilization experiments) from the second RP-HPLC separation peptide fractions were analyzed by tandem mass spectrometry. Briefly, a 10 ng/μL aliquot of the sample (dissolved in an aqueous solution of 0.1% formic acid) was infused into an Absciex QTRAP® 6500 mass spectrometer (Absciex Ltd., Foster City, Calif., USA) coupled to an electrospray ionization (ESI) source. Operating conditions were 5.5 kV ion spray voltage at 200° C., and 30 μL/min flow rate for 2 min in the positive ion mode with 2000 m/z scan maximum. MS/MS spectra were analyzed using PEAKS 7.0 Studio software (Bioinformatics Solutions, Waterloo, ON, Canada) to obtain peptide sequences. The identified peptides were chemically synthesized (>95% purity) by Genscript Inc. (Piscataway, N.J., USA).

Statistical analysis. Data analyses were performed made using one-way analysis of variance (ANOVA) with an IBM SPSS Statistical package (version 20). Mean values were compared using the Duncan Multiple Range Test and significantly differences accepted at p<0.05.

The scope of the claims should not be limited by the preferred embodiments set forth in the examples but should be given the broadest interpretation consistent with the description as a whole.

TABLE 1
Degree of Hydrolysis of Enzymatic Beef Protein Hydrolysates

| | degree of |
|---|---|
| enzyme | hydrolysis (%) |
| Alcalase ® | 35.57 ± 0.01 |
| Chymotrypsin ® | 25.83 ± 0.02 |
| Trypsin ® | 24.18 ± 0.02 |
| Pepsin ® | 8.60 ± 0.02 |
| Flavourzyme ® | 47.02 ± 0.02 |
| Thermoase ® | 18.26 ± 0.01 |

TABLE 2
Amino Acid Composition (%) of Defatted Beef
Protein (DBP) and the Enzymatic Hydrolysates*

| AA | DBP | AH | CH | PH | TH | TMH | FH | Mean ± SD | P-Value |
|---|---|---|---|---|---|---|---|---|---|
| Asx | 9.98 | 10.41 | 10.25 | 9.99 | 10.19 | 10.48 | 10.46 | 10.23 ± 0.22 | 0.02 |
| Thr | 4.63 | 4.45 | 4.67 | 4.26 | 4.43 | 4.73 | 4.36 | 4.48 ± 0.18 | 0.11 |
| Ser | 4.27 | 4.21 | 4.32 | 4.13 | 4.07 | 4.41 | 4.35 | 4.25 ± 0.13 | 0.71 |
| Glx | 15.51 | 15.95 | 16.18 | 14.30 | 16.31 | 15.89 | 17.86 | 15.94 ± 0.90 | 0.23 |
| Pro | 4.79 | 5.07 | 4.28 | 5.58 | 4.74 | 4.44 | 4.18 | 4.72 ± 0.53 | 0.75 |
| Gly | 5.46 | 6.50 | 4.34 | 7.33 | 5.56 | 5.15 | 4.57 | 5.58 ± 1.15 | 0.82 |
| Ala | 6.01 | 6.32 | 5.87 | 6.31 | 6.09 | 5.98 | 6.51 | 6.18 ± 0.24 | 0.14 |
| Cys | 1.03 | 0.89 | 0.98 | 0.89 | 0.83 | 0.91 | 0.82 | 0.89 ± 0.06 | 0.002 |
| Val | 4.55 | 4.39 | 4.57 | 4.89 | 4.34 | 4.64 | 4.57 | 4.57 ± 0.20 | 0.84 |
| Met | 2.63 | 1.81 | 2.29 | 1.88 | 1.85 | 1.89 | 2.02 | 1.96 ± 0.18 | 0.001 |
| Ile | 4.21 | 3.91 | 4.24 | 4.30 | 3.97 | 4.35 | 4.18 | 4.16 ± 0.18 | 0.51 |
| Leu | 7.86 | 7.56 | 7.95 | 7.12 | 7.56 | 7.87 | 8.07 | 7.69 ± 0.35 | 0.28 |
| Tyr | 3.35 | 3.10 | 3.49 | 2.93 | 2.96 | 3.43 | 2.72 | 3.11 ± 0.30 | 0.10 |
| Phe | 4.22 | 3.92 | 4.31 | 4.15 | 3.77 | 4.27 | 3.82 | 4.04 ± 0.23 | 0.12 |
| His | 4.13 | 4.03 | 4.26 | 4.42 | 4.40 | 4.05 | 5.05 | 4.37 ± 0.37 | 0.18 |
| Lys | 8.85 | 8.98 | 9.30 | 8.75 | 9.66 | 9.20 | 7.73 | 8.94 ± 0.67 | 0.76 |
| Arg | 6.29 | 6.59 | 6.52 | 6.80 | 7.29 | 6.31 | 6.88 | 6.72 ± 0.37 | 0.04 |
| Trp | 1.02 | 0.67 | 0.88 | 0.75 | 0.66 | 0.92 | 0.59 | 0.75 ± 0.13 | 0.004 |
| HAA | 39.66 | 37.64 | 38.86 | 38.80 | 36.75 | 38.70 | 37.48 | 38.04 ± 0.87 | 0.01 |
| PCAA | 19.27 | 19.61 | 20.08 | 19.97 | 21.34 | 19.45 | 19.66 | 20.02 ± 0.69 | 0.05 |
| NCAA | 25.49 | 26.36 | 26.43 | 24.21 | 26.50 | 26.37 | 28.32 | 26.37 ± 1.30 | 0.16 |
| AAA | 8.59 | 7.69 | 8.68 | 7.83 | 7.38 | 8.62 | 7.14 | 7.90 ± 0.64 | 0.04 |
| SCAA | 3.66 | 2.70 | 3.27 | 2.76 | 2.68 | 2.80 | 2.84 | 2.84 ± 0.22 | 0.001 |
| BCAA | 16.62 | 15.86 | 16.76 | 16.31 | 15.87 | 16.85 | 16.81 | 16.41 ± 0.47 | 0.32 |

*AH, alcalase hydrolysate; CH, chymotrypsin hydrolysate; PH, pepsin hydrolysate; TH, trypsin hydrolysate; TMH, thermoase hydrolysate; FH, flavourzyme hydrolysate HAA: hydrophobic amino acids (alanine, valine, isoleucine, leucine, tyrosine, phenylalanine, tryptophan, proline, methionine and cysteine); PCAA: positively charged amino acids (histidine, lysine, arginine); NCAA: negatively charged amino acids (Asx = asparagine + aspartic acid, Glx = glutamine + glutamic acid); AAA: aromatic amino acids (phenylalanine, tryptophan, tyrosine)
SCAA: Sulphur-containing amino acids (cysteine, methionine);
BCAA: Branched-chain amino acid (valine, isoleucine, leucine)

TABLE 3
Peptides Identified from T2R4-Inhibitory alcalase hydrolysate
(AH) and chymotrypsin hydrolysate (CH) RP-HPLC fractions

| peptide | Obs | | suggested | | | Calculated |
|---|---|---|---|---|---|---|
| Source | (m/z) | Z | peptide | parent protein | position | mol. wt. (Da) |
| AH-F1-3 | 233.1 | 2 | TMTL | Versican core protein | f529-532 | 446.6 |
| AH-F1-3 | 233.1 | 2 | ETCL | Coagulation factor XIII, | f1540-1545 | 446.5 |
| | | | | B polypeptide | | |
| AH-F1-3 | 306.2 | 2 | SSMSSL | Cardiomyopathy associated | f1540-1545 | 592.72 |
| | | | | protein 1 | | |
| AH-F1-3 | 407 | 2 | ETSARHL | Myosin class II heavy chain | f23-29 | 794.85 |
| CH-F4-3 | 509 | 2 | AGDDAPRAVF | Alpha-actin-2, Alpha-actin-1, | f24-33 | 1000.08 |
| | | | | Alpha-cardiac actin | | |
| CH-F4-5 | 228.1 | 2 | AAMY | DDR2 protein | f368-371 | 436.56 |
| | | | | FDPS protein | f280-283 | |
| CH-F4-5 | 228.1 | 2 | VSSY | Desmin, | f20-23 | 436.43 |
| | | | | Fibrillin-1 | f308-311 | |
| | | | | Glucagon | f107-110 | |
| CH-F4-5 | 228.1 | 2 | AAYM | KRT5 protein | f282-285 | 436.56 |

### REFERENCES

(1) Levit, A.; Nowak, S.; Peters, M.; Wiener, A.; Meyerhof, W.; Behrens, M.; Niv, M. Y. The bitter pill: Clinical drugs that activate the human bitter taste receptor TAS2R14. FASEB J. 2014, 28, 1181-1197.
(2) Meyerhof, W. Elucidation of mammalian bitter taste. Rev. Physiol. Biochem. Pharmacol. 2005, 154, 37-72.
(3) Upadhyaya, J.; Pydi, S. P.; Singh, N.; Aluko, R. E.; Chelikani, P. Bitter taste receptor T2R1 is activated by dipeptides and tripeptides. Biochem. Biophys. Res. Commun. 2010, 398, 331-335.
(4) Sainz, E.; Cavenagh, M. M.; Gutierrez, J.; Battey, J. F.; Northup, J. K.; Sullivan, S. L. Functional characterization of human bitter taste receptors. Biochem. J. 2007, 403, 537-43.
(5) Pydi, S. P.; Sobotkiewicz, T.; Billakanti, R.; Bhullar, R. P.; Loewen, M. C.; Chelikani, P. Amino acid derivatives as bitter taste receptor (T2R) blockers. J. Biol. Chem. 2014, 289, 25054-25066.
(6) Pydi, S. P.; Jaggupilli, A.; Nelson, K. M.; Abrams, S. R.; Bhullar, R. P.; Loewen, M. C.; Chelikani, P. Abscisic acid acts as a blocker of the bitter taste G protein-coupled receptor T2R4. Biochem. 2015, 54, 2622-2631.
(7) Roland, W. S. U.; Gouka, R. J.; Gruppen, H.; Driesse, M.; Van Buren, L.; Smit, G.; Vincken, J. P. 6-Methoxyflavanones as bitter taste receptor blockers for hTAS2R39. PLoS One 2014, 9(4), e94451.
(8) Jaggupilli, A.; Howard, R.; Upadhyaya, J. D.; Bhullar, R. P.; Chelikani, P. Bitter taste receptors: Novel insights into the biochemistry and pharmacology. Int. J. Biochem. Cell Biol., 2016, 77, 184-196.
(9) Wu, S. V.; Rozengurt, N.; Yang, M.; Young, S. H.; Sinnett-Smith, J.; Rozengurt, E. Expression of bitter taste receptors of the T2R family in the gastrointestinal tract and enteroendocrine STC-1 cells. Proc. Natl. Acad. Sci. 2002, 99, 2392-2397.
(10) Deshpande, D. A.; Wang, W. C. H.; Mcllmoyle, E. L.; Robinett, K. S.; Schillinger, R. M.; An, S. S.; Liggett, S. B. Bitter taste receptors on airway smooth muscle bronchodilate by localized calcium signaling and reverse obstruction. Nature Med. 2010, 16, 1299-1304.
(11) Zhang, C. H.; Lifshitz, L. M.; Uy, K. F.; Ikebe, M.; Fogarty, K. E.; ZhuGe, R. The cellular and molecular basis of bitter tastant-induced bronchodilation. PLoS Biology 2013, 11, e1001501.
(12) Shaik, F. A.; Singh, N.; Arakawa, M.; Duan, K.; Bhullar, R. P.; Chelikani, P. Bitter taste receptors: Extraoral roles in pathophysiology. Int. J. Biochem. Cell Biol., 2016, 77, 197-204.
(13) Kannan, A.; Hettiarachchy, N. S.; Marshall, M.; Raghavan, S.; Kristinsson, H. Shrimp shell peptide hydrolysates inhibit human cancer cell proliferation. J. Sci. Food Agric. 2011, 91, 1920-1924.
(14) Ryu, B.; Qian, Z. J.; Kim, S. K. SHP-1, a novel peptide isolated from seahorse inhibits collagen release through the suppression of collagenases 1 and 3, nitric oxide products regulated by NF-??B/p38 kinase. Peptides 2010, 31, 79-87.
(15) Udenigwe, C. C.; Aluko, R. E. Food protein-derived bioactive peptides: Production, processing, and potential health benefits. J. Food Sci. 2012, 77, R11-R24.
(16) Fuller, W.; Qurtz, R. J. Composition comprising bitter- or metallic-tasting eatable and tastand-comprising (hydroxy-, amino- or mercapto)-substituted di:carboxylic acid or derivatives, especially di:peptide containing aspartic acid. U.S. Pat. No. 5,643,955, 1997.
(17) Ley, J. P. Masking bitter taste by molecules. Chem. Percept. 2008, 1, 58-77.
(18) Lindqvist, M. Flavour improvement of water solutions comprising bitter amino acids. URL (https://stud.epsilon.slu.se/2232/1/Lindqvist_M_110201.pdf), 2010.
(19) Akitomi, H.; Tahara, Y.; Yasuura, M.; Kobayashi, Y.; Ikezaki, H.; Toko, K. Quantification of tastes of amino acids using taste sensors. Sens. Actuators B Chem. 2013, 179, 276-281.
(20) Williams, P. G. Nutritional composition of red meat. Nutr. Dietetics 2007, 64(Suppl. 4), S113-S119.
(21) Hou, H.; Li, B.; Zhao, X. Enzymatic hydrolysis of defatted mackerel protein with low bitter taste. J. Ocean Univ. China 2011, 10, 85-92.
(22) Lin, S.; Tian, W.; Li, H.; Cao, J.; Jiang, W. Improving antioxidant activities of whey protein hydrolysates obtained by thermal preheat treatment of pepsin, trypsin, alcalase and flavourzyme. Int. J. Food Sci. Technol. 2012, 47, 2045-2051.
(23) Nchienzia, H. A.; Morawicki, R. O.; Gadang, V. P. Enzymatic hydrolysis of poultry meal with endo- and exopeptidases. Poultry Sci. 2010, 89, 2273-2280.
(24) Charoenphun, N.; Cheirsilp, B.; Sirinupong, N.; Youravong, W. Calcium-binding peptides derived from tilapia (Oreochromis niloticus) protein hydrolysate. Eur. Food Res. Technol. 2013, 236, 57-63.
(25) Nielsen, P. M.; Petersen, D.; Dambmann, C. Improved method for determining food protein degree of hydrolysis. J. Food Sci. 2001, 66, 642-646.
(26) Bidlingmeyer, B. A.; Cohen, S. A.; Tarvin, T. L. Rapid analysis of amino acids using pre-column derivatization. J. Chrom. B: Biomedical Sciences and Applications 1984, 336, 93-104.
(27) Gehrke, C. W.; Wall, L. L.; Absheer, J. S.; Kaiser, F. E.; Zumwalt, R. W. Sample preparation for chromatography of amino-acids: acid hydrolysis of proteins. J. Assoc. Official Anal. Chem. 1985, 68, 811-821.
(28) Landry, J.; Delhaye, S. Simplified procedure for the determination of tryptophan of foods and feedstuffs from barytic hydrolysis. J. Agric. Food Chem. 1992, 40, 776-779.
(29) He, R.; Alashi, A.; Malomo, S. A.; Girgih, A. T.; Chao, D.; Ju, X.; Aluko, R. E. Antihypertensive and free radical scavenging properties of enzymatic rapeseed protein hydrolysates. Food Chem. 2013, 141, 153-159.
(30) Alpha MOS. Astree electrochemical sensor technology—Technical note: T-SAS-04. 2004.
(31) Girgih, A. T.; Udenigwe, C. C.; Aluko, R. E. Reverse-phase HPLC separation of hemp seed (Cannabis sativa L.) protein hydrolysate produced peptide fractions with enhanced antioxidant capacity. Plant Foods Hum. Nutr. 2013, 68, 39-46.
(32) Chuesiang, P.; Sanguandeekul, R. Protein hydrolysate from tilapia frame: antioxidant and angiotensin I converting enzyme inhibitor properties. Int. J. Food Sci. Technol. 2015, 50, 1436-1444.
(33) Wanasundara, P. K. J. P. D.; Amarowicz, R.; Pegg, R.; Shand, P. J. Preparation and characterization of hydrolyzed proteins from defibrinated bovine plasma. J. Food Sci. 2002, 67, 623-630.
(34) Kohl, S.; Behrens, M.; Dunkel, A.; Hofmann, T.; Meyerhof, W. Amino acids and peptides activate at least five members of the human bitter taste receptor family. J. Agric. Food Chem. 2013, 61, 53-60.
(35) Maehashi, K.; Huang, L. Bitter peptides and bitter taste receptors. Cell. Mol. Life Sci. 2009, 66, 1661-1671.
(36) Kim, M. R.; Choi, S. Y.; Lee, C. H. Molecular characterization and bitter taste formation of tryptic hydrolysis of 11S glycinin. J. Microbiol. Biotechnol. 1999, 9, 509-513.
(37) Rachid, O.; Simons, F. E. R.; Rawas-Qalaji, M.; Simons, K. J. An electronic tongue: Evaluation of the masking efficacy of sweetening and/or flavoring agents on the bitter taste of epinephrine. AAPS PharmSciTech. 2010, 11, 550-557.
(38) Wu, X.; Onitake, H.; Haraguchi, T.; Tahara, Y.; Yatabe, R.; Yoshida, M.; Toko, K. Quantitative prediction of bitterness masking effect of high-potency sweeteners using taste sensor. Sens. Actuators B Chem. 2016, 235, 11-17.
(39) Upadhyaya, J. D.; Chakraborty, J.; Shaik, F. A.; Jaggupilli, A.; Bhullar, R. P.; Chelikani, P. The pharmacochaperone activity of quinine on bitter taste receptors. PLoS One, 2016, 11, e0156347.
(40) Sun, X. D. Enzymatic hydrolysis of soy proteins and the hydrolysates utilization. Int. J. Food Sci. Technol. 2011, 46, 2447-2459.
(41) Tamura, M.; Mori, N.; Miyoshi, T.; Koyama, S.; Kohri, H.; Okai, H. Practical debittering using model peptides and related compounds. Agric. Biol. Chem. 1990, 54, 41-51.
(42) Humiski, L.; Aluko, R. E. Physicochemical and bitterness properties of enzymatic pea protein hydrolysates. J. Food Sci. 2007, 72, 5605-5611.
(43) Geisenhoff, H. Bitterness of soy protein hydrolysates according to molecular weight of peptides, MSc thesis dissertaion, Iowa State Univeristy, 2009.
(44) Miyanaga, Y.; Tanigake, A.; Nakamura, T.; Kobayashi, Y.; Ikezaki, H.; Taniguchi, A.; Uchida, T. Prediction of the bitterness of single, binary- and multiple-component amino acid solutions using a taste sensor. Int. J. Pharm. 2002, 248, 207-218.
(45) Rudnitskaya, A.; Nieuwoudt, H. H.; Muller, N.; Legin, A.; Du Toit, M.; Bauer, F. F. Instrumental measurement of bitter taste in red wine using an electronic tongue. Anal. Bioanal. Chem. 2010, 397, 3051-3060.
(46) Saimadi, B. H.; Ismail, A. Antioxidative peptides from food proteins: A review. Peptides 2010, 31, 1949-1956.
(47) Edens, L.; Dekker, P.; Van Der Hoeven, R.; Deen, F.; De Roos, A.; Floris, R. Extracellular prolyl endoprotease from Aspergillus niger and its use in the debittering of protein hydrolysates. J. Agric. Food Chem. 2005, 53, 7950-7957.
(48) FitzGerald, R. J.; O'Cuinn, G. Enzymatic debittering of food protein hydrolysates. Biotechnol. Adv. 2006, 24, 234-237.
(49) Singh, N.; Vrontakis, M.; Parkinson, F.; Chelikani, P. Functional bitter taste receptors are expressed in brain cells. Biochem. Biophys. Res. Commun. 2011, 406, 146-151.
(50) Xu, J.; Cao, J.; Iguchi, N.; Riethmacher, D.; Huang, L. Functional characterization of bitter-taste receptors expressed in mammalian testis. Mol. Hum. Reprod. 2013, 19, 17-28.
(51) Jaggupilli, A.; Singh, N.; Upadhyaya, J.; Sikarwar, A. S.; Arakawa, M.; Dakshinamurti, S.; Chelikani, P. Analysis of the expression of human bitter taste receptors in extraoral tissues. Mol. Cell. Biochem. 2017, 426, 137-147.
(52) Hoon, M. A., et al., Putative mammalian taste receptors: a class of taste-specific GPCRs with distinct topographic selectivity. Cell, 1999. 96(4): p. 541-51.
(53) Lagerstrom, M. C. and H. B. Schioth, Structural diversity of G protein-coupled receptors and significance for drug discovery. Nat Rev Drug Discov, 2008. 7(4): p. 339-57.
(54) Takeda, S., et al., Identification of G protein-coupled receptor genes from the human genome sequence. FEBS Lett, 2002. 520(1-3): p. 97-101.
(55) Schoneberg, T., et al., Mutant G-protein-coupled receptors as a cause of human diseases. Pharmacol Ther, 2004. 104(3): p. 173-206.
(56) Brockhoff, A., et al., Broad tuning of the human bitter taste receptor hTAS2R46 to various sesquiterpene lactones, clerodane and labdane diterpenoids, strychnine, and denatonium. J Agric Food Chem, 2007. 55(15): p. 6236-43.
(57) Born, S., et al., The human bitter taste receptor TAS2R10 is tailored to accommodate numerous diverse ligands. J Neurosci, 2013. 33(1): p. 201-13.
(58) Sakurai, T., et al., The human bitter taste receptor, hTAS2R16, discriminates slight differences in the configuration of disaccharides. Biochem Biophys Res Commun, 2010. 402(4): p. 595-601.
(59) Behrens, M., et al., The human bitter taste receptor hTAS2R50 is activated by the two natural bitter terpenoids andrographolide and amarogentin. J Agric Food Chem, 2009. 57(21): p. 9860-6.
(60) Brockhoff, A., et al., Structural requirements of bitter taste receptor activation. Proc Natl Acad Sci USA, 2010. 107(24): p. 11110-5.
(61) Zhao, D., et al., Effect of Storage on Lactase-Treated beta-Casein and beta-Lactoglobulin with Respect to Bitter Peptide Formation and Subsequent in Vitro Digestibility. J Agric Food Chem, 2017. 65(38): p. 8409-8417.

1. A method of isolating and identifying a bitter taste blocker peptide from a protein hydrolysate comprising:
providing a quantity of protein;
generating one or more hydrolysates by hydrolyzing the protein with a protease;
separating the one or more hydrolysates into fractions using a first peptide separation technique;
evaluating each respective one of the fractions for ability to antagonize a bitter taste receptor (T2R) in cell culture and selecting the most active fractions;
separating the most active fractions into sub-fractions using a second peptide separation technique;
evaluating each respective one of the sub-fractions for ability to antagonize a bitter taste receptor (T2R) in cell culture and selecting the most active sub-fractions; and
passing each respective one most active sub-fraction through a mass-spectrometer, thereby identifying a bitter taste blocker peptide.

2. The method according to claim 1 wherein the one or more hydrolysates is prepared using one or more proteases selected from the group consisting of: alcalase, thermoase, pepsin, trypsin, flavourzyme and chymotrypsin.

3. The method according to claim 1 wherein the protein is a food-derived protein.

4. A method of isolating and identifying a bitter taste blocker peptide from protein derived hydrolysate comprising:
providing a quantity of protein;
generating a first protein hydrolysate by hydrolyzing a first portion of the protein with a first protease;
generating a second protein hydrolysate by hydrolyzing a second portion of the protein with a second protein protease
generating a third protein hydrolysate by hydrolyzing a third portion of the protein with a third protease;
generating a fourth protein hydrolysate by hydrolyzing a fourth portion of the protein with a fourth protease;
separating each food protein hydrolysate into fractions using a first peptide separation technique;
evaluating each respective one of the fractions for ability to antagonize a bitter taste receptor (T2R) in cell culture and selecting the most active fractions;
separating the most active fractions into sub-fractions using a second peptide separation technique;
evaluating each respective one of the sub-fractions for ability to antagonize a bitter taste receptor (T2R) in cell culture and selecting the most active sub-fractions; and
passing each respective one most active sub-fraction through a mass-spectrometer, thereby identifying a bitter taste blocker peptide.

5. The method according to claim 4 wherein the first, second, third and fourth proteases are alcalase, thermoase, pepsin and trypsin.

6. The method according to claim 4 further comprising:
generating a fifth protein hydrolysate by hydrolyzing a fifth portion of the protein with a fifth protease; and
generating a sixth protein hydrolysate by hydrolyzing a sixth portion of the protein with a sixth protease.

7. The method according to claim 6 wherein the fifth and sixth proteases are flavourzyme and chymotrypsin.

8. The method according to claim 1 wherein the fractions and the sub-fractions are separated on a column.

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. A method of treating a food product comprising applying to said food product an effective amount of a bitter taste blocking peptide, said peptide consisting of an amino acid sequence selected from the group consisting of: TMTL (SEQ ID No:1); ETCL (SEQ ID No:2); SSMSSL (SEQ ID No:3); ETSARHL (SEQ ID No:4); AGDDAPRAVF (SEQ ID No:5); AAMY (SEQ ID No:6); VSSY (SEQ ID No:7); and AAYM (SEQ ID No:8); and a food product.

25. The method according to claim 24 wherein the food product is a food product that has associated therewith a bitter taste.

26. The method according to claim 1 wherein the one or more hydrolysates is prepared using one or more proteases selected from the group consisting of: alcalase, thermoase, pepsin, trypsin, flavourzyme, chymotrypsin, protease S, protex 6L and protex 50FP.
